(19) United States
(12) Patent Application Publication
(10) Pub. No.: US 2006/0253300 A1
(43) Pub. Date: Nov. 9, 2006

(54) System and method for managing patient triage in an automated patient management system

(76) Inventors: Benjamin L. Somberg, Shoreview, MN (US); Kenneth P. Hoyme, Plymouth, MN (US); Howard D. Simms, Shoreview, MN (US); Muralidharan Srivathsa, Shoreview, MN (US); David C. Johnson, Inver Grove Heights, MN (US)

Correspondence Address: LAW OFFICES OF PATRICK J.S. INOUYE, 810 THIRD AVE, STE. 258, SEATTLE, WA, 98104, US

(21) Appl. No.: 11/121,594

(22) Filed: May 3, 2005

(51) Int. Cl. G06Q 10/00; A61B 5/00

(52) U.S. Cl. 705/2; 600/300

(57) ABSTRACT

A system and method for managing patient triage in an automated patient management system is presented. A criteria for placement of patient data into a display is defined for a plurality of remotely managed patients. The patient data originates from one or more patient data sources operating on each such patient and selected from at least one of a physiological sensor and a therapy delivery device. An ordering of the patient data within the display is defined based on a need of care in relation to one or more of a type of health condition, severity of the health condition, and facilities available to attend to the health condition. An organization of the patient data within the display is defined in relation to metadata associated with the patient data.

### FIELD OF THE INVENTION

The present invention relates in general to automated patient management and, specifically, to a system and method for managing patient triage in an automated patient management system.

### BACKGROUND OF THE INVENTION

In general, medical devices and sensors, including implantable medical devices (IMDs), such as pacemakers or defibrillators, provide therapy delivery, such as pacing, cardiac resynchronization, defibrillation, neural stimulation, and drug delivery, as well as physiological data monitoring. These devices and sensors function autonomously by relying on preprogrammed operation and control over therapeutic and monitoring functions, but must still be periodically interfaced for follow-up to external devices, such as programmers and similar devices, to check status, for programming and troubleshooting, and to download telemetered patient data.

The currency and amount of patient data available is dependent upon the frequency of follow-up. Normally, follow-up occurs in-clinic once every three to twelve months, or as necessary. Although mandatory, the clinic visits are often the only one-on-one interpersonal contact that occurs between the patient and his or her physician, absent complications or other health-related issues. Other follow-up methods, such as transtelephonic monitoring, can enable a healthcare provider to remotely interrogate devices and sensors on a monthly basis. More recently, remote dedicated patient monitoring devices, known as repeaters, have enabled healthcare providers to perform follow-up monitoring on a daily basis using a data communications network, such as the Internet.

Such frequent monitoring has significantly increased the amount of patient data potentially available due to the substantially continuous monitoring that many such devices and sensors are capable of providing. Sifting through collected patient data presents a difficult and time-consuming task, which includes identifying those patients presenting with potential health issues, as well as ensuring therapy compliance and performing routine reviews of detectable non-critical conditions. This problem is exacerbated as the number of remotely managed patients continues to increase.

Therefore, there is a need for an approach to efficiently identifying and prioritizing remotely managed patients in need of medical care through collected patient data analysis. Preferably, such an approach would balance considered medical need with pragmatic clinical practice considerations.

### SUMMARY OF THE INVENTION

A system and method includes forming an ordered and prioritized list of remotely managed patients. Patient data is collected from patient data sources, including implantable and external medical devices and sensors. The patient data is evaluated against a criteria specified by a clinician to determine whether a patient will be placed on the patient list. Selected patients are prioritized using a triage that factors in health condition types, health condition severities, and available facilities. Finally, metadata is applied to organize the patient list.

One embodiment provides a system and method for managing patient triage in an automated patient management system. A criteria for placement of patient data into a display is defined for a plurality of remotely managed patients. The patient data originates from one or more patient data sources operating on each such patient and selected from at least one of a physiological sensor and a therapy delivery device. An ordering of the patient data within the display is defined based on a need of care in relation to one or more of a type of health condition, severity of the health condition, and facilities available to attend to the health condition. An organization of the patient data within the display is defined in relation to metadata associated with the patient data.

Still other embodiments of the present invention will become readily apparent to those skilled in the art from the following detailed description, wherein are described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a functional block diagram showing, by way of example, an automated patient management environment, in accordance with one embodiment.

FIG. 2 is a functional block diagram showing data collection in the environment of FIG. 1.

FIG. 3 is a data flow diagram showing patient triage as implemented in the environment of FIG. 1.

FIG. 4 is a process flow diagram showing patient selection and prioritization in the environment of FIG. 1.

FIG. 5 is a data flow diagram showing patient selection and prioritization settings for use in the environment of FIG. 1.

FIG. 6 is a screen diagram showing, by way of example, a patient list display generated by the server of FIG. 1.

FIG. 7 is a functional block diagram showing a server for managing patient triage in an automated patient management system for use in the environment of FIG. 1.

### DETAILED DESCRIPTION

Automated Patient Management Environment

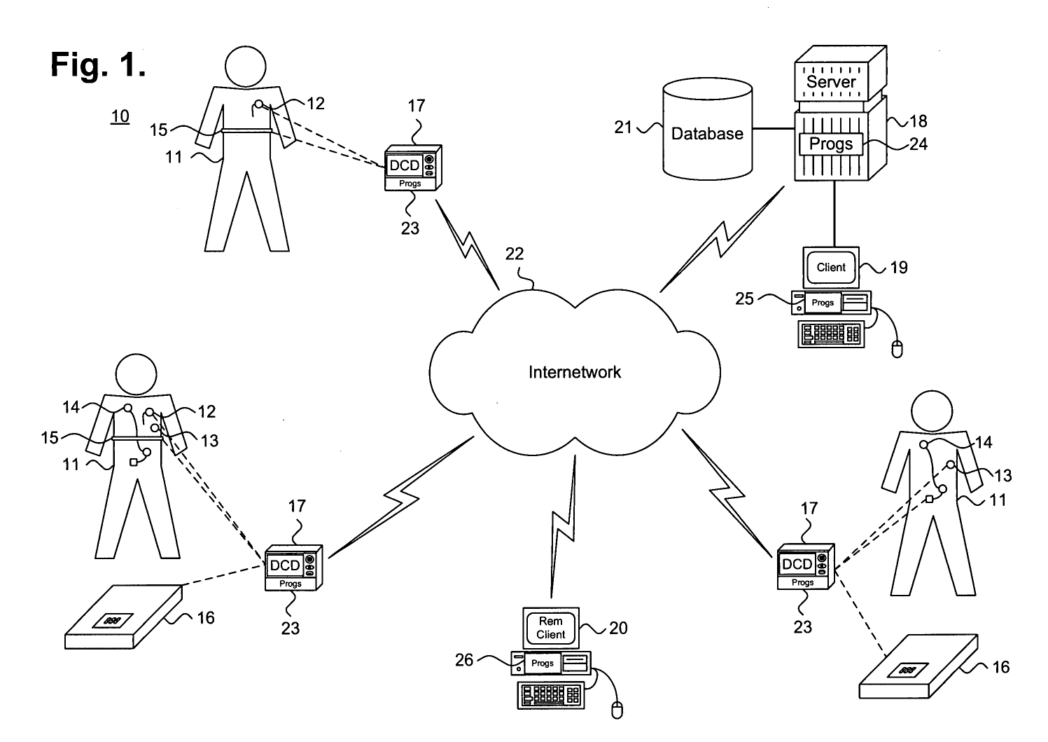

Automated patient management encompasses a range of activities, including remote patient management and automatic diagnosis of patient health, such as described in commonly-assigned U.S. Patent application Pub. No. US 2004/0103001, pending, published May 27, 2004, the disclosure of which is incorporated by reference. Such activities can be performed proximal to a patient, such as in the patient's home or office, through a centralized server, such as in a hospital, clinic or physician's office, or through a remote workstation, such as a secure wireless mobile computing device. FIG. 1 is a functional block diagram showing, by way of example, an automated patient management environment 10, in accordance with one embodiment. A plurality of individual patients 11 are remotely managed through one or more data collection devices 17, for example, such as described in commonly-assigned U.S. patent application, entitled, “System and Method for Managing Alert Notifications in an Automated Patient Management System,” Ser. No. ______, filed May 3, 2005, pending, the disclosure of which is incorporated by reference. Each data collection device 17 is interconnected remotely over an internetwork 22, such as the Internet to a centralized server 18. The internetwork 22 can provide both conventional wired and wireless interconnectivity. In one embodiment, the internetwork 22 is based on the Transmission Control Protocol/Internet Protocol (TCP/IP) network communication specification, although other types or combinations of networking implementations are possible. Similarly, other network topologies and arrangements are possible.

Each data collection device 17 is uniquely assigned to an individual patient 11 to provide a localized and network-accessible interface to one or more patient data sources 12-16, either through direct means, such as wired connectivity, or through indirect means, such as inductive, radio frequency or wireless telemetry based on, for example, “strong” Bluetooth or IEEE 802.11 wireless fidelity “WiFi” interfacing standards. Other configurations and combinations of patient data source interfacing are possible.

Patient data includes physiological measures, which can be quantitative or qualitative, parametric data regarding the status and operational characteristics of the patient data source itself, and environmental parameters, such as the temperature or time of day. Other types of patient data are possible. The patient data sources collect and forward the patient data either as a primary or supplemental function. Patient data sources include, by way of example, medical devices that deliver or provide therapy to the patient 11, sensors that sense physiological data in relation to the patient 11, and measurement devices that measure environmental parameters occurring independent of the patient 11. Each patient data source can generate one or more types of patient data and can incorporate one or more components for delivering therapy, sensing physiological data and measuring environmental parameters. In a further embodiment, data values could be entered by a patient 11 directly into a patient data source. For example, answers to health questions could be input into a measurement device that includes interactive user interfacing means, such as a keyboard and display or microphone and speaker. Such patient-provided data values could also be collected as patient information. Additionally, measurement devices are frequently incorporated into medical devices and sensors. Medical devices include implantable medical devices (IMDs) 12, such as pacemakers, implantable cardiac defibrillators (ICDs), drug pumps, and neuro-stimulators, and external medical devices (EMDs) 14, such as automatic external defibrillators (AEDs). Sensors include implantable sensors 13, such as implantable heart and respiratory monitors and implantable diagnostic multi-sensor non-therapeutic devices, and external sensors 15, 16, such as Holter monitors, weight scales, and blood pressure cuffs. Other types of medical, sensing, and measuring devices, both implantable and external, are possible.

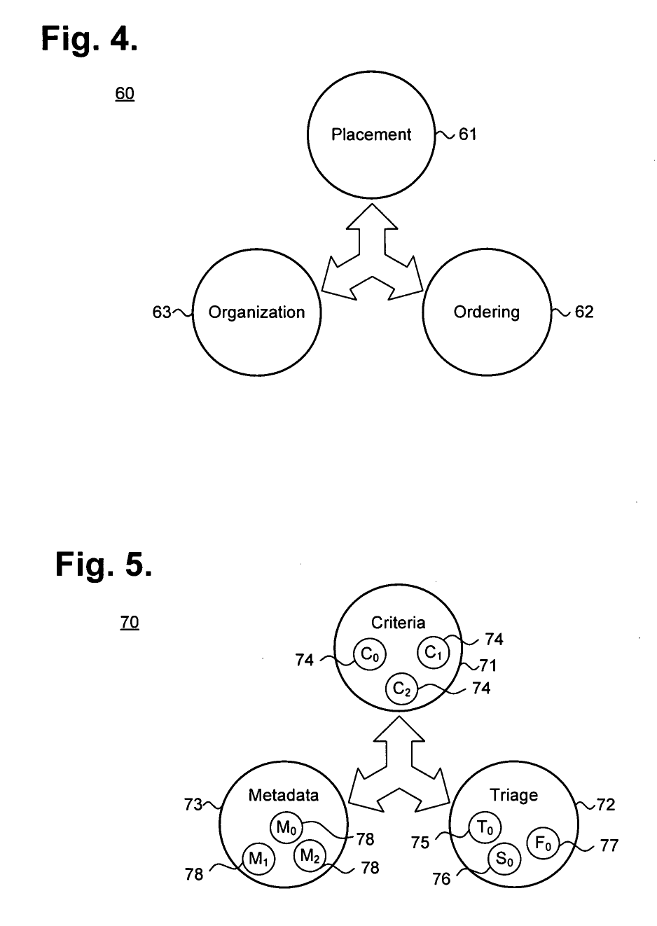

The data collection device 17 collects and temporarily stores patient data from the patient data sources 12-16 for periodic upload over the internetwork 22 to the server 18 and storage in a database 21. Patient data collection can be defined to be initiated by either the patient collection device 17 or by one or more of the patient data sources 12-16. The collected patient data can also be selected and prioritized by one or more locally-configured clients 19 or one or more remote clients 20 securely-interconnected over the internetwork 22, as further described below with reference to FIGS. 4 and 5. Access to the collected patient data includes a capability to provide flexible display and control that is securely and intelligently coordinated between a plurality of clinicians, such as physicians, nurses, or qualified medical specialists. In a further embodiment, the clients 19 and remote clients 20 can be used, for example, by clinicians, such as physicians, nurses, or qualified medical specialists, to securely access stored patient data assembled in the database 21, such as described in commonly-assigned U.S. patent application, entitled, “System and Method for Managing Coordination of Assembled Patient Data in an Automated Patient Management System,” Ser. No. ______, filed May 3, 2005, pending, the disclosure of which is incorporated by reference. Although described herein with reference to clinicians, the entire discussion applies equally to organizations, including hospitals, clinics, and laboratories, and other individuals or interests, such as researchers, scientists, universities, and governmental agencies, seeking access to the patient data.

The collected patient data can also be evaluated for the occurrence of one or more conditions, such as described in related, commonly-owned U.S. Pat. No. 6,336,903, to Bardy, issued Jan. 8, 2002; U.S. Pat. No. 6,368,284, to Bardy, issued Apr. 9, 2002; U.S. Pat. No. 6,398,728, to Bardy, issued Jun. 2, 2002; U.S. Pat. No. 6,411,840, to Bardy, issued Jun. 25, 2002; and U.S. Pat. No. 6,440,066, to Bardy, issued Aug. 27, 2002, the disclosures of which are incorporated by reference. Patient data evaluation can be defined to be performed by either the patient collection device 17 or the server 18.

Finally, conditions occurring in the collected patient data can trigger one or more alert notifications that provide external indicators of the condition occurrences. Alert notification can be defined to be performed by either the server 18, patient collection device 17, or one or more other devices either operating as part of or as an adjunct to the internetwork 22.

In a further embodiment, patient data is safeguarded against unauthorized disclosure to third parties, including during collection, assembly, evaluation, transmission, and storage, to protect patient privacy and comply with recently enacted medical information privacy laws, such as the Health Insurance Portability and Accountability Act (HIPAA) and the European Privacy Directive. At a minimum, patient health information that identifies a particular individual with health- and medical-related information is treated as protectable, although other types of sensitive information in addition to or in lieu of specific patient health information could also be protectable.

Preferably, the server 18 is a server-grade computing platform configured as a uni-, multi- or distributed processing system, and the clients 19 and remote clients 20 are general-purpose computing workstations, such as a personal desktop or notebook computer. In addition, the data collection device 17, server 18, clients 19, and remote clients 20 are programmable computing devices that respectively execute set of software programs 23, 24, 25, 26 and include components conventionally found in computing device, such as, for example, a central processing unit (CPU), memory, network interface, persistent storage, and various components for interconnecting these components.

Data Collection

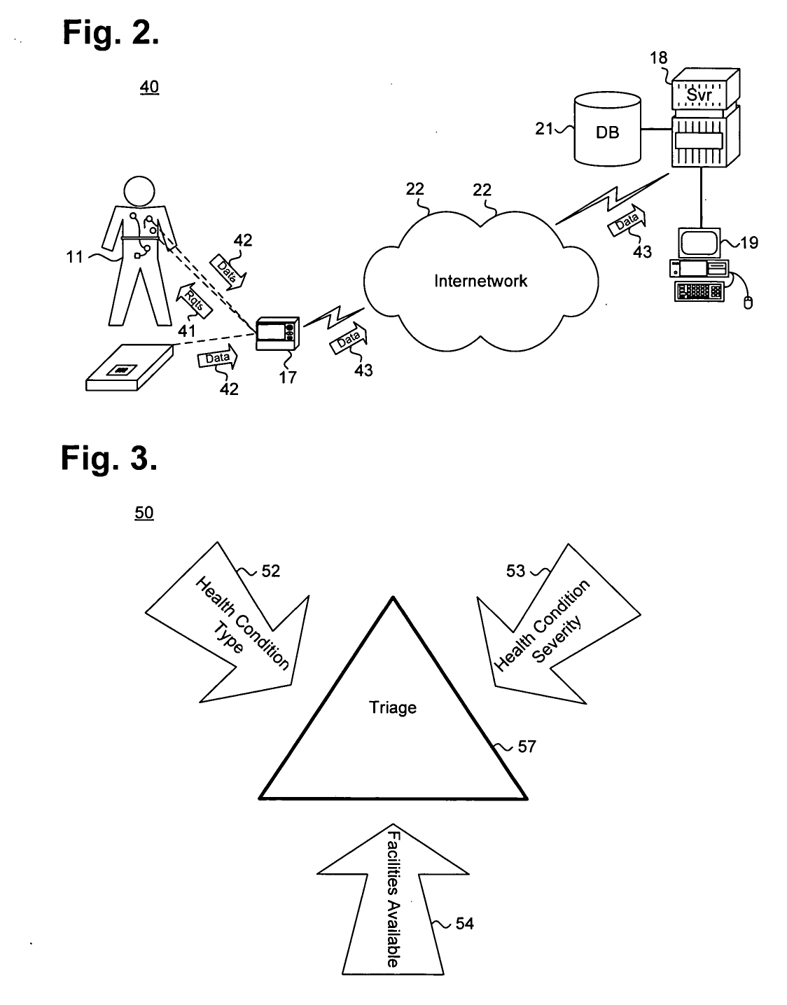

Automated patient management allows a potentially enormous amount of patient data to be generated for each patient 11 through substantially continuous monitoring, which consequently requires careful selection and prioritization of patients, also referred to as “triage,” as further described below with reference to FIG. 3, to ensure timely and prudent provisioning of medical care. FIG. 2 is a functional block diagram showing data collection 40 in the environment 10 of FIG. 1. The data collection process reflects the dichotomy of data collection device-versus patient data source-initiated data collection.

Patient data sources that operate autonomously from the patient are generally able to record patient data at any time and under any conditions. However, the recorded patient data accumulated by the patient data source must be periodically uploaded to free limited onboard storage and to facilitate processing and analysis. In one embodiment, schedules can be associated with a subset of the interfaced patient data sources to provide data collection device-initiated patient data collection. Alternatively, a schedule can also be provided to initiate prompted retrieval of patient data by the remotely managed patient. A schedule might be appropriate for a patient data source, such as an implanted cardiac pulse generator, where patient data may be collected on a daily or weekly basis. The schedule can either be built into the data collection device 17 or can be provided by the server 18, based on configuration options selected by the clinician. The data collection device attempts to collect patient data at a scheduled interval by sending requests 41 to the associated patient data source, which returns patient data 42. In the absence of expected patient data receipt, the data collection device 17 can implement a follow-up scheme with the patient data source, if possible, to investigate delayed or missing patient data, or by sending a message or other communication to the patient 11, clinician or authorized third party as a compliance notification.

Scheduled data collection might not be appropriate for all patient data sources 12-16. For example, a battery powered weight scale that uses radio frequency telemetry to communicate with a data collection device 17 would normally be turned off to extend battery life. Ordinarily, such a device would communicate with the data collection device 17 only after use by the patient and would otherwise be in a standby or sleep state. Such devices frequently operate in a send-only mode and may therefore be incapable of receiving incoming messages. The patient data source asynchronously sends patient data 42 to the data collection device 17 to provide patient data source-initiated patient data collection. In one embodiment, frequencies can be associated with a subset of the interfaced patient data sources to allow the data collection device 17 to track or limit the receipt of patient data. In the absence of expected patient data receipt, the data collection device 17 can implement a follow-up scheme with the patient data source, if possible, to investigate delayed or missing patient data, or by sending a message or other communication to the patient 11, clinician or authorized third party as a compliance notification.

Finally, collected patient data 43 is uploaded by the data collection device 17 to the server 18 either upon receipt or, in a further embodiment, after a delay to allow patient data 42 from multiple patient data sources to accumulate. The server 18 stores the uploaded patient data 43 in the database 21 as collected patient data.

Patient Triage

Medical resources are finite and not every patient that presents for medical care requires immediate attention. Rather, prudent medical practice allows clinicians to select and prioritize patients based on their need of care. FIG. 3 is a data flow diagram showing patient triage 57 as implemented in the environment 10 of FIG. 1. “Triage” refers to the sorting of patients in need of care based on three factors, which include the type of health condition 52 of which the patient is complaining or presents, the severity of the health condition 53, and the facilities available 54 for providing medical care. The weight assigned to each of these factors need not be assigned equally. Rather, the type and severity of the health condition, for instance, may take a higher precedence over available facilities in a large metropolitan emergency room. Other types and weightings of factors are possible.

Patient Selection and Prioritization

In the context of automated patient management of a population of remotely managed patients 11, triage enables a clinician to prudently consider the large volume of patient data generated by the substantially continuous monitoring and reporting of patient data from patient data sources 12-16 and to identify those patients 11 in need of some form of medical care. FIG. 4 is a process flow diagram showing patient selection and prioritization 60 in the environment 10 of FIG. 1. The process involves ensuring that each set of reported patient data is properly evaluated to identify an appropriate course of health care provisioning.

In one embodiment, patient placement 61, ordering 62, and organization 63 are provided using the server 18. Generally, patient data is collected from patient data sources 12-16 by data collection devices 17 for eventual upload to the server 18. Once received, the server 18 stores the collected patient data into the database 21 to be made available for clinician use. In a further embodiment, the collected patient data is also analyzed by the server 18 to determine patient wellness, device status, and similar information.

Patient placement 61 is used to establish a criteria for placing remotely managed patients 11 on a patient list. Those patients that are not placed on the patient list must still be documented. Both in-clinic and remote medical care can be provided, as well as chronicling of confirming patient compliance and the routine review of detectable non-critical conditions. In addition, each clinician can establish an appropriate criteria to ensure that the medical needs of multiple clinicians are accommodated. Other patient placement criteria are possible.

Ordering 62 specifies the location that remotely managed patients 11 are placed on a prioritized patient list. In one embodiment, triage 57 is applied to order those patients 11 that are placed on the patient list and the weights assigned to each factor are determined by the clinician as appropriate. Other types of orderings are possible.

Finally, organization 63 allows a clinician to factor in other considerations bearing on medical care provisioning. The considerations are specified as metadata that identify factors typically not bearing on patient placement or ordering, although metadata could also overlap with those considerations. Other types of organizations are possible.

Patient Selection and Prioritization Settings

Clinician considerations effecting patient list management are specified as a set of settings maintained by the server 18. FIG. 5 is a data flow diagram showing patient selection and prioritization settings 70 for use in the environment of FIG. 1. The settings specify criteria 71, triage 72, and metadata 73 considerations that respectively facilitate patient list placement, ordering, and organization.

In one embodiment, a patient placement criteria 71 is specified by defining one or more individual criterion 74. As a minimum criteria for patient list placement, a patient must be remotely managed through the operation of a data collection device 17 that is operatively interfaced to one or more patient data sources 12-16. Other criteria considerations are possible.

Triage considerations 72 are specified by assigning weights or priorities to specific types or classes of health conditions 75, the severity of the health condition presented 76, and the facilities available to the clinician 77 to care for the patient. Other triage considerations are also possible.

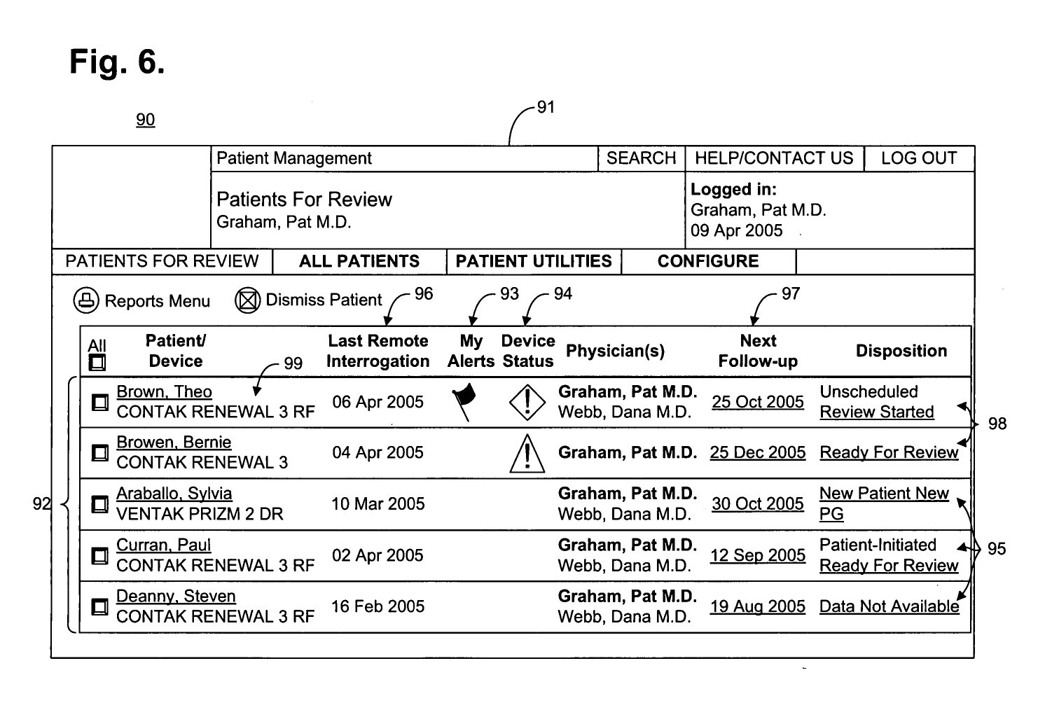

Finally, metadata 73 is specified as a set of individual loosely-related metadata considerations 78, as further described below with reference to FIG. 6. For instance, metadata 73 can relate to specific indicators or parameters found in the collected patient data, such as device status flags, or to generalized clinic issues, such as workflow management or external data sources. Other metadata considerations are possible.

In a further embodiment, a clinician can determine whether other clinicians have been interacting with the patients on the patient list. The current disposition of each patient record includes a list identifying all clinicians that have historically reviewed that patient record. Other types of multi-clinician information provisioning are possible.

Sample User Interface

In one embodiment, clinician-customizable displays are provided as viewable pages in a Web-based format, although other types of formats, as well as physical media, are possible. FIG. 6 is a screen diagram 90 showing, by way of example, a patient list display 91 generated by the server 18 of FIG. 1. The patient list display 91 can be viewed via a Web browser executing on the clients 19, remote clients 20, or other compatible computing systems. Moreover, although described with reference to a viewable Web page, patient list displays can also include other formats and physical media. The patient list display 91 includes a set of patient records 92 that includes a summary of collected patient data. Other types of information can also be included.

The following metadata considerations are provided for patient list organization:
Alerts 93: includes indicators of the overall status of the patient or medical device or sensor, specific health indicators, such as weight gain, indicators obtained from interfaced medical devices, such as high lead impedance, or indicators obtained from interfaced sensors. Can be interrelated to non-customizable indicators 94.
Non-Customizable Indicators 94: includes non-customizable indicators that are made available to all clinicians. Can be interrelated to alerts 93.
Disposition 95: includes recently-added patients or patient data originating from a patient-initiated interrogation of a patient data source.
Last Remote Interrogation 96: includes a prioritized list of patients, ordered by length of time since a last data collection episode.
Next Follow-Up 97: includes the date of a next follow-up appointment, which can permit rescheduling, for instance, if the clinician is unavailable.
Workflow States 98: allows a clinician to ensure that high priority patients are being reviewed promptly and that the clinician is informed of the review status of each patient.
Workflow Parameters: allows filtering or prioritizing based on clinic-related considerations, such as billing predisposition, electronic medical record exportation, referral letter generation, and follow-up letter generation.
External Sources 99: includes events occurring in relation to an external data source, such as events in other databases. For instance, an external database could supply notifications related to specific implanted device models effected by a recall notice or research findings. Can also include other types of uncategorized information, such as clinician notes, annotations, or attachments.
   Other metadata considerations are possible.
   Server

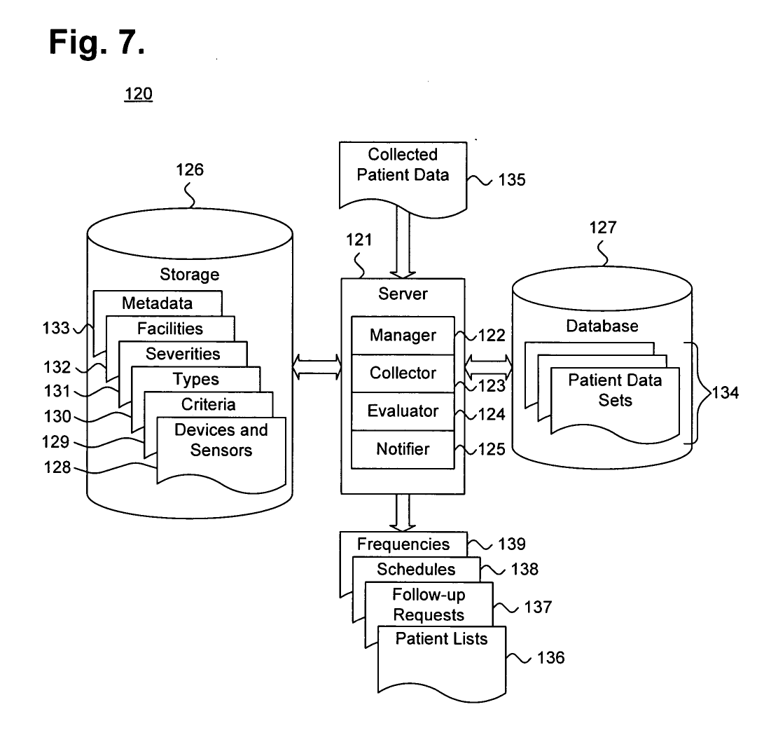

The server acts as the central hub for selecting and prioritizing patient care. FIG. 7 is a functional block diagram 120 showing a server 121 for managing patient triage in an automated patient management system for use in the environment 10 of FIG. 1. The server 121 includes storage 126 and database 127 and can be configured to coordinate the displaying of patient data for multiple patients between a plurality of clients 19, remote clients 20, and other compatible computing systems. Other server functions are possible.

At a minimum, the server 121 includes a manager 122. The manager 122 selects patients for placement on a patient list based on criteria 129 specified by a clinician. The manager 122 orders the selected patients through triage that factors in health condition types 130, health condition severities 131, and available facilities 131. Finally, the manager 122 applies metadata 133 to organize the patient list.

In a further embodiment, the server 121 further includes a collector 123, evaluator 124, and notifier 125. The collector 123 maintains a list of devices and sensors 128 for all patient data sources 12-16, which can be used by a clinician to create schedules 138 and maximum frequencies 139 to manage the collection of patient data by interfaced data collection devices. The collector 123 collects patient data 135 received from the data collection devices, which are stored as patient data sets 134 in the database 127. The collector 123 can execute a follow-up scheme, for example, by sending follow-up requests 137 to patient data sources, if possible, that have not sent expected collected patient data, or by sending a message or other communication to the patient 11, clinician or authorized third party as a compliance notification.

The evaluator 124 evaluates the collected patient data 135 against a complete set of alert conditions. One or more triggers are associated with the alert conditions and occurrences of alert condition set off the associated triggers. The same alert conditions can be evaluated by both the server 121 and one or more of the data collection devices.

The notifier 125 provides alert notifications. Alert notifications are assigned to notification schemes that are executed upon the detection of an associated trigger. The notification schemes can be organized into one or more levels of alerts. By way of example, alert notifications 164 can include a Web page update, phone or pager call, E-mail, SMS, text or “Instant” message, as well as a message to the patient send through the data collection device 17 and simultaneous direct notification to emergency services and to the clinician. Other alert notifications are possible.

While the invention has been particularly shown and described as referenced to the embodiments thereof, those skilled in the art will understand that the foregoing and other changes in form and detail may be made therein without departing from the spirit and scope of the invention.

What is claimed is:

1. A system for managing patient triage in an automated patient management system, comprising:
a criteria defined for placement of patient data into a display for a plurality of remotely managed patients, wherein the patient data originates from one or more patient data sources operating on each such patient and selected from at least one of a physiological sensor and a therapy delivery device;
an ordering of the patient data defined within the display based on a need of care in relation to one or more of a type of health condition, severity of the health condition, and facilities available to attend to the health condition; and
an organization of the patient data defined within the display in relation to metadata associated with the patient data.

2. A system according to claim 1, wherein the metadata is selected from the group comprising an indicator of overall status of the patient or device, specific health indicators or indicators obtained from one or more of the patient data sources.

3. A system according to claim 1, wherein the metadata is selected from the group comprising an indicator of a condition occurring in relation to at least one such patient data.

4. A system according to claim 1, wherein the metadata is selected from the group comprising recently-added patients or patient data originating from a patient-initiated interrogation of a patient data source.

5. A system according to claim 1, wherein the metadata is selected from the group comprising a prioritized list of patients ordered by length of time since last data collection episode.

6. A system according to claim 1, wherein the metadata is selected from the group comprising a date of next follow-up appointments for patients.

7. A system according to claim 1, wherein the metadata is selected from the group comprising workflow state to ensure that high priority patients are being reviewed promptly and review status of each patient to ensure that a clinician is properly informed.

8. A system according to claim 1, wherein the metadata is selected from the group comprising workflow parameters relating to one or more of billing predisposition, electronic medical record exportation, referral letter generation, and follow-up letter generation.

9. A system according to claim 1, wherein the metadata is selected from the group comprising events occurring in relation to an external data source.

10. A system according to claim 1, further comprising:
one or more triggers defined to be associated with a condition occurring in relation to at least one such patient data evaluateable subsequent to collection; and
a notification scheme determined to be executable upon detection of at least one such trigger to provide an external indicator of the condition occurrence.

11. A system according to claim 1, further comprising:
a database to store the collected patient data, wherein the stored collected patient data is included in relation to the placement of patient data into a display.

12. A system according to claim 1, wherein the patient data source comprises at least one of an implantable medical device, implantable diagnostic multi-sensor non-therapeutic device, external medical device, implantable sensor, and external sensor.

13. A method for managing patient triage in an automated patient management system, comprising:
defining a criteria for placement of patient data into a display for a plurality of remotely managed patients, wherein the patient data originates from one or more patient data sources operating on each such patient and selected from at least one of a physiological sensor and a therapy delivery device;
defining an ordering of the patient data within the display based on a need of care in relation to one or more of a type of health condition, severity of the health condition, and facilities available to attend to the health condition; and
defining an organization of the patient data within the display in relation to metadata associated with the patient data.

14. A method according to claim 13, wherein the metadata is selected from the group comprising an indicator of overall status of the patient or device, specific health indicators or indicators obtained from one or more of the patient data sources.

15. A method according to claim 13, wherein the metadata is selected from the group comprising an indicator of a condition occurring in relation to at least one such patient data.

16. A method according to claim 13, wherein the metadata is selected from the group comprising recently-added patients or patient data originating from a patient-initiated interrogation of a patient data source.

17. A method according to claim 13, wherein the metadata is selected from the group comprising a prioritized list of patients ordered by length of time since last data collection episode.

18. A method according to claim 13, wherein the metadata is selected from the group comprising a date of next follow-up appointments for patients.

19. A method according to claim 13, wherein the metadata is selected from the group comprising workflow state to ensure that high priority patients are being reviewed promptly and review status of each patient to ensure that a clinician is properly informed.

20. A method according to claim 13, wherein the metadata is selected from the group comprising workflow parameters relating to one or more of billing predisposition, electronic medical record exportation, referral letter generation, and follow-up letter generation.

21. A method according to claim 13, wherein the metadata is selected from the group comprising events occurring in relation to an external data source.

22. A method according to claim 13, further comprising:
defining one or more triggers associated with a condition occurring in relation to at least one such patient data evaluateable subsequent to collection; and
determining a notification scheme executable upon detection of at least one such trigger to provide an external indicator of the condition occurrence.

23. A method according to claim 13, further comprising:
storing the collected patient data; and
including the stored collected patient data in relation to the placement of patient data into a display.

24. A method according to claim 13, wherein the patient data source comprises at least one of an implantable medical device, implantable diagnostic multi-sensor non-therapeutic device, external medical device, implantable sensor, and external sensor.

25. A computer-readable storage medium holding code for performing the method according to claim 13.

26. An apparatus for managing patient triage in an automated patient management system, comprising:
means for defining a criteria for placement of patient data into a display for a plurality of remotely managed patients, wherein the patient data originates from one or more patient data sources operating on each such patient and selected from at least one of a physiological sensor and a therapy delivery device;
means for defining an ordering of the patient data within the display based on a need of care in relation to one or more of a type of health condition, severity of the health condition, and facilities available to attend to the health condition; and
means for defining an organization of the patient data within the display in relation to metadata associated with the patient data.
